(19) United States
(12) Patent Application Publication
(10) Pub. No.: US 2015/0355181 A1
(43) Pub. Date: Dec. 10, 2015

(54) METHODS AND COMPOSITIONS FOR HUMAN EPIDIDYMIS PROTEIN-4 (HE4)

(71) Applicant: BETH ISRAEL DEACONESS MEDICAL CENTER, INC., Boston, MA (US)

(72) Inventors: Raghu KALLURI, Weston, MA (US); Valerie S. LEBLEU, Brookline, MA (US)

(21) Appl. No.: 14/763,202

(22) PCT Filed: Jan. 24, 2014
(86) PCT No.: PCT/US14/12924

§ 371 Date: Jul. 24, 2015

Related U.S. Application Data
(60) Provisional application No. 61/756,855, filed on Jan. 25, 2013.

(51) Int. Cl. G01N 33/573; A61K 45/06; A61K 39/395; A61K 31/7088

(52) U.S. Cl. CPC G01N 33/573; A61K 31/7088; A61K 45/06; A61K 39/395; G01N 2333/96433; G01N 2500/10; G01N 2500/04

(57) ABSTRACT

The present invention relates to methods, compositions, and diagnostic tests for treating and diagnosing a subject with organ fibrosis or a risk of developing organ fibrosis. The present invention also relates to methods and compositions for treating a subject with a proliferative disease. In particular, the methods and compositions include treatment of organ fibrosis or a proliferative disease using an inhibitor of human epididymis protein-4 (HE4).

### CROSS-REFERENCE TO RELATED APPLICATIONS

This application claims the benefit of U.S. Provisional Application No. 61/756,855 filed Jan. 25, 2013, which is hereby incorporated by reference in its entirety.

### STATEMENT AS TO FEDERALLY FUNDED RESEARCH

This work was supported by grant number NCI-CA 125550 from the National Cancer Institute. The Government has certain rights in the invention.

### BACKGROUND OF THE INVENTION

Renal fibrosis is the scarring and chronic pathological remodeling of the kidney in which the normal tissue architecture is progressively replaced by type I collagen and other extracellular matrix proteins. Accumulation of type I collagen leads to structural and functional alterations of the kidney parenchyma and eventual organ failure. Most chronic renal damage, irrespective of etiology, leads to renal fibrosis, a self-perpetuating process that is probably facilitated by the recruitment of activated fibroblasts (myofibroblasts) and the progression of an inflammatory response. Many previous studies have suggested a possible role for myofibroblasts in the production of scar-forming type I collagen and the pathogenesis of renal fibrosis.

HE4 encodes for a highly conserved WAP domain-containing protein, which is suggestive of putative serine protease inhibitor activity. The protein is implicated in sperm maturation and potentially has a role in natural immunity, but the biological function of HE4 is unknown. Interestingly, HE4 was identified as the most upregulated gene in fibrotic kidneys of dogs. HE4 was also reported to be significantly upregulated in fibrotic kidneys of mice, and its transcript level in human kidney transplant biopsies was found to be strongly correlated with low estimated glomerular filtration rate (eGFR). Despite these observations, the role of HE4 in renal fibrosis and its putative serine protease activity has remained unexplored.

Consequently, there exists a need in the art to identify new biomarkers for predicting renal fibrosis and, in general, new targets for treating organ fibrosis and facilitating organ regeneration and repair.

### SUMMARY OF THE INVENTION

The invention features a method of treating a subject having organ fibrosis, the method including administering to the subject an inhibitor of human epididymis protein-4 (HE4) in an amount sufficient to treat the organ fibrosis. The invention also features a method of treating a subject having a proliferative disease, the method including administering to the subject an inhibitor of HE4 in an amount sufficient to treat said proliferative disease.

In one aspect, the method of treating a subject having organ fibrosis includes: a) determining the type I collagen content in a sample from the subject, and b) administering to a subject having increased type I collagen content an inhibitor of HE4 in an amount sufficient to treat the organ fibrosis.

In a second aspect, the method of treating organ fibrosis includes administering an inhibitor of HE4 in a subject and: a) determining the level of HE4 in a sample from the subject b) adjusting the dose of the inhibitor of HE4 in an amount sufficient to treat the organ fibrosis, wherein an improvement in renal fibrosis measures results in the treatment of the organ fibrosis.

In a third aspect, the inhibitor of HE4 is administered with a second agent. In some embodiments, the second agent is an anticancer agent or an immunosuppressive agent.

In a fourth aspect, the inhibitor of HE4 results in an increase in serine protease activity in a fibrotic organ.

In a fifth aspect, the improvement in renal fibrosis measures is selected from the group consisting of: a decrease in Masson's Trichrome staining, a decrease in type I collagen content, an increase in type I collagen digestion activity, an increase in serine protease activity, and reduced macrophage infiltration.

In a particular embodiment, the organ fibrosis is selected from the group consisting of: renal fibrosis, pulmonary fibrosis, cirrhosis, endomyocardial fibrosis, Chrohn's disease, liver fibrosis, heart fibrosis, scleroderma, and progressive massive fibrosis. In another embodiment, the proliferative disease is selected from the group consisting of: leukemia, brain cancer, bladder cancer, breast cancer, cervical cancer, colorectal cancer, endometrial cancer, esophageal cancer, head and neck cancer, liver cancer, lung cancer, lymphoma, ovarian cancer, pancreatic cancer, prostate cancer, renal cancer, skin cancer, stomach cancer, testis cancer, thyroid cancer, and urothelial cancer.

In another embodiment, the inhibitor of HE4 is administered in an amount sufficient to further facilitate organ regeneration and repair.

The invention also features a method of diagnosing a subject as having, or having a risk of developing organ fibrosis, the method including: a) obtaining a sample from said subject, b) measuring the level of HE4 in a sample from the subject, and c) comparing the level to a normal reference sample, wherein an increase in the HE4 levels compared to the normal reference sample results in diagnosing the subject as having, or having a risk of developing organ fibrosis.

In certain aspects, the invention further includes measuring the level of Prss23 or Prss35 in a sample from the subject, wherein an increase in the Prss23 or Prss35 levels compared to a normal reference sample results in diagnosing the subject as having, or having a risk of developing organ fibrosis. In another aspect, the invention includes administering to the subject an inhibitor of HE4, in an amount sufficient to treat the organ fibrosis

Finally, the invention also features a method for identifying an inhibitor of HE4, the method including contacting a cell with a candidate compound and measuring HE4 activity, wherein the presence of a decrease level of HE4 activity in the cell, as compared to a normal reference sample, identifies an inhibitor of HE4.

In all embodiments of the invention, the inhibitor of HE4 is an RNAi agent, a small molecule inhibitor, or an antibody.

### DEFINITIONS

By “amount sufficient” of an agent is meant the amount of the agent sufficient to effect beneficial or desired result (e.g., treatment of organ fibrosis, a risk of developing organ fibrosis, or a proliferative disease), and, as such, an amount sufficient of the formulation is an amount sufficient to achieve a reduction in the expression level and/or activity of the HE4 gene or protein, as compared to the response obtained without administration of the composition.

By “inhibitor of HE4” is meant an agent or compound that decreases or reduces HE4 gene expression, protein expression, or activity (e.g., enzymatic activity), as defined herein, compared to a control (e.g., a decrease by 5%, 10%, 15%, 20%, 25%, 30%, 35%, 40%, 45%, 50%, 55%, 60%, 65%, 70%, 75%, 80%, 85%, 90%, or 95%, as compared to a control or a normal reference sample). Inhibitors of HE4 can be identified and tested by any useful method (e.g., any described herein) known in the art.

By “facilitate organ regeneration and repair” is meant an inhibitor of HE4 can be used, e.g., to increase tissue function through, e.g., a decrease in fibrous tissue formation, a decrease in type I collagen content, and increase in type I collagen digestion activity, an increase in serine protease activity, and reduced macrophage infiltration.

By “increase level of HE4” is meant an increase in HE4 gene expression, protein expression, or activity (e.g., enzymatic activity), as compared to a control from a normal cell or normal tissue (e.g., an increase of at least 2-fold, e.g., from about 2-fold to about 150-fold, e.g., from 5-fold to 150-fold, from 5-fold to 100-fold, from 10-fold to 150-fold, from 10-fold to 100-fold, from 50-fold to 150-fold, from 50-fold to 100-fold, from 75-fold to 150-fold, or from 75-fold to 100-fold, as compared to a control or a normal reference sample). Increase level of activity can be determined using any useful methods known in the art or described herein. For example, an increase level of activity can be determined as an increase in HE4 gene expression or increase in HE4 protein concentration (e.g., as determined by PCR or gel electrophoresis), as compared to a control (e.g., a sample including normal cell or normal tissue from one or more healthy subjects) or a normal reference sample, as defined herein. In another example, an increase level of activity can be determined as an increase in HE4 enzymatic activity, such as by measuring a decrease in serine protease activity (e.g., from 3-fold to 4-fold decreased activity) as compared to a control or a normal reference sample. Increased level of activity can be measured directly (e.g., increased HE4 gene expression or increased HE4 enzymatic activity) or indirectly, such as by measuring activity levels of one or more proteins associated with increased HE4 activity (e.g., a serine protease e.g., trypsin, MMP2, MMP9, Prss35, and/or Prss23 e.g., from 2-fold to 4-fold, e.g., about 3-fold, decreased levels of activity) as compared to a control or a normal reference sample.

By “reference sample” is meant any sample, standard, standard curve, or level that is used for comparison purposes. A “normal reference sample” can be, for example, a prior sample taken from the same subject; a sample from a normal healthy subject; a sample from a subject not having a disease associated with increased levels of HE4, a sample from a subject that is diagnosed with a propensity to develop organ fibrosis or a proliferative disease associated with increased levels of HE4, but does not yet show symptoms of the disorder; a sample from a subject that has been treated for organ fibrosis or a proliferative disease; or a sample of purified HE4 at a known normal concentration.

By “RNAi agent” is meant any agent or compound that exerts a gene silencing effect by hybridizing a target nucleic acid. RNAi agents include any nucleic acid molecules that are capable of mediating sequence-specific RNAi (e.g., under stringent conditions), for example, a short interfering RNA (siRNA), double-stranded RNA (dsRNA), microRNA (miRNA), short hairpin RNA (shRNA), short interfering oligonucleotide, short interfering nucleic acid, short interfering modified oligonucleotide, short interfering nucleic acid, short interfering modified oligonucleotide, chemically-modified siRNA, post-transcriptional gene silencing RNA (ptgsRNA), and Dicer-substrate RNA (DsiRNA).

By “proliferative disease” is meant a condition characterized by rapidly dividing cells resulting in uncontrolled growth of new tissue, parts, and/or surrounding cells.

Other features and advantages of the invention will be apparent from the following Detailed Description, the drawings, and the claims.

### BRIEF DESCRIPTION OF THE DRAWINGS

FIGS. 1A-1G show the accumulation of αSMA+ myofibroblasts in the interstitium and expression of HE4 in renal fibrosis. FIG. 1A is a visualization of αSMA-RFP+ cells in control (contralateral kidney to UUO) and fibrotic mouse kidneys (UUO) from αSMA-RFP transgenic mice. DAPI (light colored), nuclei. Also shown is a quantification of αSMA+ cells per visual field in the control and fibrotic kidney at day 10 after UUO. Scale bar, 50 μm. FIG. 1B is a visualization of αSMA-RFP+ myofibroblasts cultured from control kidneys (mouse normal fibroblasts, mNFs) and fibrotic kidneys (mouse FAFs, mFAFs). Scale bars, 50 μm. FIG. 1C is a graph showing the relative HE4 gene expression in mFAFs normalized to that in mNFs. AU, arbitrary units. FIG. 1D is a western blot analysis of HE4 in mNFs and mFAFs. Actin was used as an internal control. rHE4, recombinant HE4 protein used as positive control. FIG. 1E is a western blot analysis for HE4 in mNF and mFAF culture media with loading normalized to cell numbers. BSA was also used to control for lane loading. FIG. 1F is a graph showing relative HE4 gene expression in control kidneys (n=5) and fibrotic kidneys (n=5) evaluated at day 10 after UUO. FIG. 1G is a Western blot analysis of HE4 in mouse control and fibrotic kidneys. Actin was used as an internal control. Data are shown as the mean±s.e.m. *P<0.05 determined by t test.

FIGS. 2A-2H are results showing that HE4 is a pan-serine protease and an MMP2 and MMP9 inhibitor that prevents type I collagen degradation. FIG. 2A is a graph showing pNA release assessed by a spectrophotometric readout of color development as a measure of serine protease activity in nonfibrotic control kidney lysates, fibrotic kidney lysates (at day 10 after UUO) and fibrotic kidney lysates (at day 10 after UUO) incubated with recombinant HE4 protein (rHE4). FIG. 2B is a graph showing relative Prss23 and Prss35 gene expression in mouse FAFs (mFAFs) and mouse fibrotic kidneys (Fibrotic) compared and normalized to mouse normal fibroblasts (mNF; derived from nonfibrotic kidneys) and contralateral nonfibrotic kidneys (evaluated at day 10 after UUO), respectively. mNFs and contralateral nonfibrotic kidneys were set arbitrarily to a value of 1 (white bar). AU, arbitrary units. FIG. 2C is a graph showing pNA release assessed by a spectrophotometric readout of color development using Prss23 and Prss35 serine proteases with and without rHE4. FIG. 2D is a graph showing hydroxyproline release assay showing free hydroxyproline from type I collagen (Coll) digestion and type I collagen digested by Prss23 or Prss35 with and without rHE4 and with and without antibody to HE4 (anti-HE4). FIG. 2E is a graph showing trypsin activity in nonfibrotic control kidney lysates, fibrotic (at day 10 after UUO) kidneys and fibrotic kidneys pretreated with rHE4. FIG. 2F is a graph showing hydroxyproline release assay showing free hydroxyproline from type I collagen digestion and type I collagen digested by trypsin with and without HE4, with and without BSA and with and without the antibody to HE4. FIG. 2G is a graph showing hydroxyproline release assay showing free hydroxyproline from type I collagen digestion and type I collagen digested by MMP2 or MMP9 with and without rHE4 and with and without the antibody to HE4. FIG. 2H is an immunoprecipitation (IP) blot of fibrotic kidney lysates using IgG (control) or antibody to HE4 and western blot analysis for MMP2 and MMP9. Data are shown as the mean±s.e.m. *P<0.05 determined by t test.

FIGS. 3A-3F are data showing HE4 neutralization inhibits kidney fibrosis. FIG. 3A show representative Masson trichrome (MTS) and type I collagen staining of control and fibrotic kidneys from mice treated with antibody to HE4 (UUO, n=5; NTN, n=6) or IgG (UUO, n=5; NTN, n=5) showing reduced fibrosis in mice treated with antibody to HE4 (anti-HE4). AU, arbitrary units. Scale bars, 50 μm. FIG. 3B are graphs showing blood urea nitrogen (BUN) and urine albumin-to-creatinine ratio (U-albumin/U-creatinine) measurements of mice with NTN treated with IgG (n=5) or antibody to HE4 (n=6). FIG. 3C shows immunolabeling for HE4 and αSMA in fibrotic kidneys from mice treated with antibody to HE4 or IgG. DAPI (light colored dots), nuclei. The histogram shows the relative number of αSMA+, HE4+ and αSMA+HE4+(double positive) cells per field of view. Scale bar, 50 μm. FIG. 3D is a gel showing gelatin zymography using lysates of contralateral (control) kidneys, fibrotic kidneys of mice treated with IgG and fibrotic kidneys of mice treated with antibody to HE4. Lysates are from kidneys at day 10 after UUO. The histogram shows the relative band intensities normalized to actin. MW, molecular weight; HT1080, positive control lysates. FIG. 3E is a graph showing serine protease activity (left) and trypsin activity (right) in kidney lysates (UUO) from control kidneys, fibrotic kidneys of mice treated with IgG and fibrotic kidneys of mice treated with antibody to HE4 (left). Lysates are from kidneys at day 10 after UUO. FIG. 3F shows Western blot analyses for MMP2, MMP9 and the actin loading control of lysates of contralateral (control) kidneys of mice treated with IgG or antibody to HE4 and fibrotic kidneys of mice treated with IgG or antibody to HE4. Lysates are from kidneys at day 10 after UUO. The histogram shows the relative band intensities normalized to actin. Data are shown as the mean±sem. *P<0.05, NS, not significant determined by t test.

FIGS. 4A-4F are data showing that HE4 is elevated in human fibrotic kidneys, human FAFs and serum of patients with renal fibrosis. FIG. 4A is a graph showing relative HE4 gene expression of different fibroblast cultures (normalized to normal fibroblasts (NF1, line TK173) set arbitrarily to a value of 1). The normal fibroblast lines used were as follows: NF1, TK173; NF2, TK231a; NF3, TK163. The FAF lines used (FAF1-FAF6) were as follows: FAF1, TK274; FAF2, TK188; FAF3, TK239; FAF4, TK261; FAF5, TK257. AU, arbitrary units. FIG. 4B is a graph showing relative PRSS23 and PRSS35 gene expression of different fibroblast cultures (normalized to normal fibroblasts set arbitrarily to 1). FIG. 4C shows a Western blot analysis of HE4 in human normal fibroblasts (from left to right (NF1-NF3), lines TK173, TK231a and TK163) and human FAFs (from left to right (FAF1-FAF3), lines TK274, TK188 and TK239). Actin was used as an internal control. FIG. 4D shows a Western blot analysis of HE4 in human normal fibroblast and FAF culture media with loading normalized to cell numbers. BSA was also used to control for lane loading. FIG. 4E shows immunolabeling for HE4 and αSMA in human kidneys with chronic kidney disease (Alport syndrome) and renal fibrosis. DAPI (light colored), nuclei. Arrows indicate cells double positive for αSMA and HE4. Scale bar, 50 μm. (f) sHE4 concentrations from healthy control individuals (n=5) and patients with chronic kidney disease (CKD) (n=11). *P<0.05 determined by t test.

### DETAILED DESCRIPTION

In general, the invention features the treatment of organ fibrosis or a proliferative disease by administering an inhibitor of human epididymis protein-4 (HE4). In certain embodiments, the inhibitor of HE4 is useful in facilitating organ regeneration and repair. The invention also features HE4 and two specific serine proteases, Prss23 and Prss35 as a diagnostic marker for organ fibrosis or a risk of developing organ fibrosis, in particular renal fibrosis. In the examples set forth below, HE4 is shown to be robustly expressed by myofibroblasts, and its concentration in the serum of patients with kidney diseases correlates with renal fibrosis. In certain embodiments, two new specific serine proteases (Prss23 and Prss35) that are significantly upregulated in kidney fibrosis, that act, in part as type I collagen-degrading enzymes and targets of HE4 protease inhibition were identified. In particular examples set forth below, HE4 was shown to inhibit degradation of type I collagen induced by matrix metalloproteinase 2 (MMP2) and MMP9 and this inhibition was reversed by use of a neutralizing antibody to HE4.

### Conditions and Disorders

The methods and compositions of the invention include administration of one or more inhibitors of HE4 (e.g., those described herein) to a subject having organ fibrosis or a proliferative disease.

Exemplary organ fibrosis include but are not limited to: renal fibrosis, pulmonary fibrosis, cirrhosis, endomyocardial fibrosis, Chrohn's disease, liver fibrosis, heart fibrosis, scleroderma, and progressive massive fibrosis.

Exemplary proliferative diseases include non-solid cancers and solid cancers, such as leukemia (e.g., chronic myeloid leukemia, acute myeloid leukemia, acute lymphoblastic leukemia, and chronic lymphocytic leukemia), brain cancer (e.g., ependymoma, glioma, medulloblastoma, meningioma, teratoid rhabdoid tumor, and teratoma), bladder cancer (e.g., adenocarcinoma, sarcoma, small cell carcinoma, squamous cell carcinoma, and transitional cell carcinoma), breast cancer (e.g., breast ductal carcinoma), cervical cancer, colorectal cancer, endometrial cancer, esophageal cancer (e.g., adenocarcinoma and squamous cell carcinoma), head and neck cancer, liver cancer (e.g., hepatocellular carcinoma, cholangiocarcinoma, and hemangioendothelioma), lung cancer (e.g., non-small cell lung cancer, small-cell lung cancer, carcinoid, sarcoma, squamous cell cancer, adenocarcinoma, and large cell carcinoma), lymphoma (e.g., malignant lymphoma), ovarian cancer (e.g., ovarian epithelial carcinoma and teratoma), pancreatic cancer, prostate cancer (e.g., adenocarcinoma and prostatic intraepithelial neoplasia), renal cancer, skin cancer (e.g., basal cell carcinoma, squamous cell carcinoma, and malignant melanoma), stomach cancer, testis cancer, thyroid cancer, and urothelial cancer.

### Diagnostic Methods

Increased levels of HE4 can also be used for the diagnosis of organ fibrosis or a risk of developing organ fibrosis. A subject having organ fibrosis or a risk of developing organ fibrosis will show an increased level of HE4 (e.g., an alteration, e.g., an increase of 10%, 20%, 30%, 40%, 50%, 60%, 70%, 80%, 90%, or more) in the expression or biological activity of HE4.

In one example, an increase in HE4 gene or protein expression or HE4 enzymatic activity, as compared to a normal reference sample or control, is indicative of organ fibrosis or a risk of developing the same. In another example, an increase in HE4 gene or protein expression or HE4 enzymatic activity is determined indirectly, such as an increase in type I collagen (e.g., a 10%, 20%, 30%, 40%, 50%, 70%, 90% increase) content, a decrease in serine protease activity (e.g., a decrease of 10%, 20%, 30%, 40%, 50%, 70%, 90%), a decrease in collagenase activity (e.g., a decrease of 10%, 20%, 30%, 40%, 50%, 70%, 90% in activity of matrix metalloproteinases such as MMP2 and MMP9, Prss35, and/or Prss23), a decrease in trypsin activity (e.g., a decrease of 10%, 20%, 30%, 40%, 50%, 70%, 90%), when compared to a control or a normal reference sample.

Standard methods may be used to measure analyte levels or cellular parameters in any bodily fluid, including, but not limited to, urine, blood, serum, plasma, saliva, or cerebrospinal fluid. Such methods include immunoassay, ELISA, Western blotting using antibodies directed to HE4 and quantitative enzyme immunoassay techniques. ELISA assays are the preferred method for measuring polypeptide levels. Accordingly, the measurement of antibodies specific to HE4 in a subject may also be used for the diagnosis of organ fibrosis or a risk of developing the same.

In one embodiment, a subject having organ fibrosis or a risk of developing the same will show an increase in the expression of a nucleic acid encoding HE4. Methods for detecting such alterations are standard in the art. In one example Northern blotting or real-time PCR is used to detect mRNA levels. In another embodiment, hybridization at high stringency with PCR probes that are capable of detecting HE4, including genomic sequences, or closely related molecules, may be used to hybridize to a nucleic acid sequence derived from a subject having organ fibrosis or a risk of developing the same. The specificity of the probe, whether it is made from a highly specific region, e.g., the 5′-regulatory region, or from a less specific region, e.g., a conserved motif, and the stringency of the hybridization or amplification (maximal, high, intermediate, or low), determine whether the probe hybridizes to a naturally occurring sequence, allelic variants, or other related sequences. Hybridization techniques may be used to identify mutations in a nucleic acid molecule, or may be used to monitor expression levels of a gene encoding a polypeptide of the invention.

Diagnostic methods can include measurement of absolute levels of a polypeptide, or nucleic acid. In any of the diagnostic methods, the level of a polypeptide or nucleic acid, or any combination thereof, can be measured at least two different times from the same subject and an alteration in the levels (e.g., by 10%, 20%, 30%, 40%, 50%, 60%, 70%, 80%, 90%, or more) over time is used as an indicator of organ fibrosis or a risk of developing the same. It will be understood by the skilled artisan that for diagnostic methods that include comparing of the polypeptide, or nucleic acid to a reference level, particularly a prior sample taken from the same subject, a change over time with respect to the baseline level can be used as a diagnostic indicator of organ fibrosis, or a predisposition to develop the same. The diagnostic methods described herein can be used individually or in combination with any other diagnostic method described herein for a more accurate diagnosis of the presence of, severity of, or predisposition to organ fibrosis or a risk of developing the same.

The diagnostic methods can also be used to track the progression of a disease and/or effectiveness of a particular treatment, e.g., levels can be used to adjust (e.g., increase or decrease) the dosages of an inhibitor of HE4.

### Inhibitors of HE4

Inhibitors of HE4 include one or more agents or compounds that directly or indirectly inhibit HE4 gene expression, protein expression, or enzymatic activity. Exemplary inhibitors of HE4 include an RNAi agent, (e.g., a shRNA for HE4), an anti-HE4 antibody, and small molecule inhibitors. Additional inhibitors of HE4 can be identified by any useful method known in the art.

### RNAi Agents

Inhibitors of HE4 include one or more RNAi agents that inhibit HE4 gene expression in a cell in vitro or in vivo (e.g., in a subject). The RNAi agents can include different types of double-stranded molecules that include either RNA:RNA or RNA:DNA strands. These agents can be introduced to cells in a variety of structures, including a duplex (e.g., with or without overhangs on the 3′-terminus), a hairpin loop, or an expression vector that express one or more polynucleotides capable of forming a double-stranded polynucleotide alone or in combination with another polynucleotide.

Exemplary RNAi agents include siRNA, shRNA, DsiRNA, and miRNA agents. Generally these agents are about 10 to about 40 nucleotides in length, and preferred lengths for particular RNAi agents include siRNA that are double-stranded RNA molecules of 16 to 30 nucleotides in length (e.g., 18 to 25 nucleotides, e.g., 21 nucleotides); shRNA that are single-stranded RNA molecules in which a hairpin loop structure is present and a stem length is between 19 to 29 nucleotides in length (e.g., 19 to 21 nucleotides or 25 to 29 nucleotides) or a loop size is between 4 to 23 nucleotides in length; DsiRNA that are double-stranded RNA agents of 25 to 35 nucleotides in length; and miRNA that are single-stranded RNA molecules of 17 to 25 nucleotides (e.g., 21 to 23 nucleotides) in length.

The RNAi agent can have any useful nucleic acid sequence, including a nucleic acid sequence having one or more DNA molecules, RNA molecules, or modified forms (e.g., a modified backbone composition or 2′-deoxy, or 2′-O-methyl modifications) or combinations thereof. Additionally, the RNAi agent can contain 5′- and/or 3′-terminal modifications and include blunt and overhanging nucleotides at these termini, or combinations thereof. Exemplary modifications include a 5′-dideoxythymidine overhang, such as for siRNAi; a 3′-UU or 3′-dTdT overhang, such as for shRNA; one or more G-U mismatches between the two strands of the shRNA stem; or a single-stranded nucleotide overhang at the 3′-terminal of the antisense or sense strand of 1 to 4 nucleotides (e.g., 1 or 2 nucleotides) for DsiRNA.

Methods of producing antisense and sense nucleotides, as well as corresponding duplexes or hairpin loops, are known in the art and can be readily adapted to produce an antisense oligonucleotide that targets any target nucleic acid sequence. RNAi agents include at least one antisense nucleotide sequence that is directed to a target nucleic acid (e.g., a target gene, e.g., a HE4 gene). Antisense nucleotides are single strands of DNA or RNA that are complementary to a chosen target sequence. In the case of antisense RNA, they prevent translation of complementary RNA strands by binding to it. Antisense DNA can be used to target an antisense nucleotides contain from about 10 to about 40 nucleotides, more preferably about 15 to about 30 nucleotides. The antisense nucleotide can have up to 80%, 85%, 90%, 95%, 99%, or even 100% complementary to the desired target gene.

### Anti-HE4 Antibodies

Inhibitors of HE4 also include one or more anti-HE4 antibodies. Exemplary antibodies include those described in U.S. Pat. No. 7,846,692, which is incorporated by reference herein in its entirety. Antibodies are also commercially available from Abcam (Cambridge, Mass.), Atlast Antibodies AB (Stockholm, Sweden, Novus Biologicals (Littleton, Colo.), LifeSpan Biosciences (Seattle, Wash.), and Santa Cruz Biotechnology (Santa Cruz, Calif.).

The invention also encompasses the generation of humanized antibodies to HE4. Various methods for humanizing non-human antibodies are known in the art. For example, a humanized antibody can have one or more amino acid residues introduced into it from a source which is non-human. These non-human amino acid residues are often referred to as “import” residues, which are typically taken from an “import” variable domain. Humanization can be essentially performed by substituting hypervariable region sequences for the corresponding sequences of a human antibody. Accordingly, such “humanized” antibodies are chimeric antibodies, where substantially less than an intact human variable domain has been substituted by the corresponding sequence from a non-human species. In practice, humanized antibodies are typically human antibodies in which at least some hypervariable region residues as well as other variable region residues are substituted by residues from analogous sites in rodent antibodies.

The choice of human variable domains, both light and heavy, to be used in making the humanized antibodies can be important to reduce antigenicity. According to the so-called “best-fit” method, the sequence of the variable domain of a rodent antibody is screened against the entire library of known human variable-domain sequences. The human sequence which is closest to that of the rodent is then accepted as the human framework for the humanized antibody. Another method uses a particular framework derived from the consensus sequence of all human antibodies of a particular subgroup of light or heavy chains. The same framework may be used for several different humanized antibodies.

It is further generally desirable that antibodies be humanized with retention of high affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, human framework region (FR) residues can be selected and combined from the recipient and import sequences so that the desired antibody characteristic, such as increased affinity for the target antigen(s), is achieved. In general, the hypervariable region residues are directly and most substantially involved in influencing antigen binding.

Human antibodies can be constructed by combining Fv clone variable domain sequence(s) selected from human-derived phage display libraries with known human constant domain sequences(s). Alternatively, human monoclonal antibodies of the invention can be made by the hybridoma method. Human myeloma and mouse-human heteromyeloma cell lines for the production of human monoclonal antibodies have been described.

It is possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge.

Gene shuffling can also be used to derive human antibodies from non-human, e.g., rodent, antibodies, where the human antibody has similar affinities and specificities to the starting non-human antibody. According to this method, which is also called “epitope imprinting,” either the heavy or light chain variable-region of a non-human antibody fragment obtained by phage display techniques as described herein is replaced with a repertoire of human V domain genes, creating a population of non-human chain/human chain scFv or Fab chimeras. Selection with antigen results in isolation of a non-human chain/human chain chimeric scFv or Fab where the human chain restores the antigen binding site destroyed upon removal of the corresponding non-human chain in the primary phage display clone, i.e., the epitope governs (imprints) the choice of the human chain partner. When the process is repeated in order to replace the remaining non-human chain, a human antibody is obtained. Unlike traditional humanization of non-human antibodies by CDR grafting, this technique provides completely human antibodies, which have no FR or CDR residues of non-human origin.

Anti-HE4 antibodies can features antibody fragments that comprise a portion of an intact antibody, preferably comprising the antigen binding region thereof. Examples of antibody fragments include Fab, Fab′, F(ab′)₂, and Fv fragments; diabodies; linear antibodies; single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

Fv is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three hypervariable regions (HVRs) of each variable domain interact to define an antigen-binding site on the surface of the VH-VL dimer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

Single-chain Fv or scFv antibody fragments comprise the V_{H} and V_{L} domains of antibody, where these domains are present in a single polypeptide chain. Generally, the scFv polypeptide further comprises a polypeptide linker between the V_{H} and V_{L} domains which enables the scFv to form the desired structure for antigen binding.

Diabodies are antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies may be bivalent or bispecific.

Antibody fragments may be generated by traditional means, such as enzymatic digestion, or by recombinant techniques. In certain circumstances there are advantages of using antibody fragments, rather than whole antibodies. The smaller size of the fragments allows for rapid clearance, and may lead to improved access to solid tumors. Various techniques have been developed for the production of antibody fragments. Traditionally, these fragments were derived via proteolytic digestion of intact antibodies. However, these fragments can now be produced directly by recombinant hosT cells. Fab, Fv, and ScFv antibody fragments can all be expressed in and secreted from E. coli, thus allowing the facile production of large amounts of these fragments. Antibody fragments can be isolated from the antibody phage libraries. Alternatively, Fab′-SH fragments can be directly recovered from E. coli and chemically coupled to form F(ab′)₂ fragments. In another approach, F(ab′)₂ fragments are isolated directly from recombinant hosT cell culture. Other techniques for the production of antibody fragments will be apparent to the skilled practitioner.

### Other Inhibitors of HE4

Small molecule inhibitors and peptide inhibitors of HE4 can include one or more compounds that inhibit HE4 activity. Screening assays can be used to identify one or more small molecule inhibitors of HE4. Candidate compounds can be tested for their effect on HE4 activity using any particular cell based assays described herein. Standard methods may be used to measure analyte levels or cellular parameters in any bodily fluid, including, but not limited to, urine, blood, serum, plasma, saliva, or cerebrospinal fluid. Such methods include immunoassay, ELISA, Western blotting using antibodies directed to HE4 and quantitative enzyme immunoassay techniques. ELISA assays are the preferred method for measuring polypeptide levels. Accordingly, the measurement of antibodies specific to HE4 in a subject may also be used to determine if a compound has effects on HE4 activity.

In one embodiment, a compound that affects HE4 activity may show a decrease in the expression of a nucleic acid encoding HE4. Methods for detecting such alterations are standard in the art. In one example Northern blotting or real-time PCR is used to detect mRNA levels. In another embodiment, hybridization techniques may be used to monitor expression levels of a gene encoding a polypeptide of the invention upon treatment with a candidate compound. In a further embodiment, a reporter gene such as a gene encoding GFP or luciferase can be fused to the HE4 promoter to monitor the expression levels of HE4 upon treatment with a candidate compound.

In general, candidate compounds are identified from large libraries of both natural product or synthetic (or semi-synthetic) extracts, chemical libraries, or from polypeptide or nucleic acid libraries, according to methods known in the art. Those skilled in the field of drug discovery and development will understand that the precise source of test extracts or compounds is not critical to the screening procedure(s) of the invention.

### Combination Therapy

The methods and compositions include combinations of an inhibitor of HE4 and a therapeutic agent, such as an anticancer agent. Exemplary anticancer agents include chemotherapeutic agents (e.g., arsenic trioxide, cisplatin, carboplatin, chlorambucil, melphalan, nedaplatin, oxaliplatin, triplatin tetranitrate, satraplatin, imatinib, nilotinib, dasatinib, and radicicol), immunomodulatory agents (e.g., methotrexate, leflunomide, cyclophosphamide, cyclosporine A, minocycline, azathioprine, antibiotics (e.g., tacrolimus), methylprednisolone, corticosteroids, steroids, mycophenolate mofetil, rapamycin, mizoribine, deoxyspergualin, brequinar, T cell receptor modulators, and cytokine receptor modulators), antiangiogenic agents (e.g., bevacizumab, suramin, and etrathiomolybdate), mitotic inhibitors (e.g., paclitaxel, vinorelbine, docetaxel, abazitaxel, ixabepilone, larotaxel, ortataxel, tesetaxel, vinblastine, vincristine, vinflunine, and vindesine), nucleoside analogs (e.g., gemcitabine, azacitidine, capecitabine, carmofur, cladribine, clofarabine, cytarabine, decitabine, floxuridine, fludarabine, fluorouracil, mercaptopurine, pentostatin, tegafur, and thioguanine), DNA intercalating agents (e.g., doxorubicin, actinomycin, bleomycin, mitomycin, and plicamycin), topoisomerase inhibitors (e.g., irinotecan, aclarubicin, amrubicin, belotecan, camptothecin, daunorubicin, epirubicin, etoposide, idarubicin, mitoxantrone, pirarubicin, pixantrone, rubitecan, teniposide, topotecan, valrubicin, and zorubicin), folate antimetabolites (e.g., pemetrexed, aminopterin, methotrexate, pralatrexate, and raltitrexed), and other targeting agents (e.g., agents that target particular enzymes in a metabolic pathway or proteins involved in cancer or agents that target particular organs or types of cancers), and combinations thereof.

The methods and compositions can also include combinations of an inhibitor of HE4 and an immunosuppressive agent. Examples of immunosuppressants include, but are not limited to, calcineurin inhibitors (e.g., cyclosporin A (Sandimmune®), cyclosporine G tacrolimus (Prograf®, Protopic®)), mTor inhibitors (e.g., sirolimus (Rapamune®, Neoral®), temsirolimus (Torisel®), zotarolimus, and everolimus (Certican®)), fingolimod (Gilenya™), myriocin, alemtuzumab (Campath®, MabCampath®, Campath-1H®), rituximab (Rituxan®, MabThera®), an anti-CD4 monoclonal antibody (e.g., HuMax-CD4), an anti-LFA1 monoclonal antibody (e.g., CD11a), an anti-LFA3 monoclonal antibody, an anti-CD45 antibody (e.g., an anti-CD45RB antibody), an anti-CD19 antibody (see, e.g., U.S. Patent Publication 2006/0280738), monabatacept (Orencia®), belatacept, indolyl-ASC (32-indole ether derivatives of tacrolimus and ascomycin), azathioprine (Azasan®, Imuran®), lymphocyte immune globulin and anti-thymocyte globulin [equine] (Atgam®), mycophenolate mofetil (Cellcept®), mycophenolate sodium (Myfortic®), daclizumab (Zenapax®), basiliximab (Simulect®), cyclophosphamide (Endoxan®, Cytoxan®, Neosar™, Procytox™ Revimmune™), prednisone, prednisolone, leflunomide (Arava®), FK778, FK779, 15-deoxyspergualin (DSG), busulfan (Myleran®, Busulfex®), fludarabine (Fludara®), methotrexate (Rheumatrex®, Trexall®), 6-mercaptopurine (Purinethol®), 15-deoxyspergualin (Gusperimus), LF15-0195, bredinin, brequinar, and muromonab-CD3 (Orthoclone®).

### EXAMPLES

The invention can be illustrated by the following non-limiting examples. These examples are set forth merely for illustrative purposes and many other variations may be used.

### Example 1

### αSMA+ Myofibroblasts Accumulate in the Interstitium and Express HE4 in Renal Fibrosis

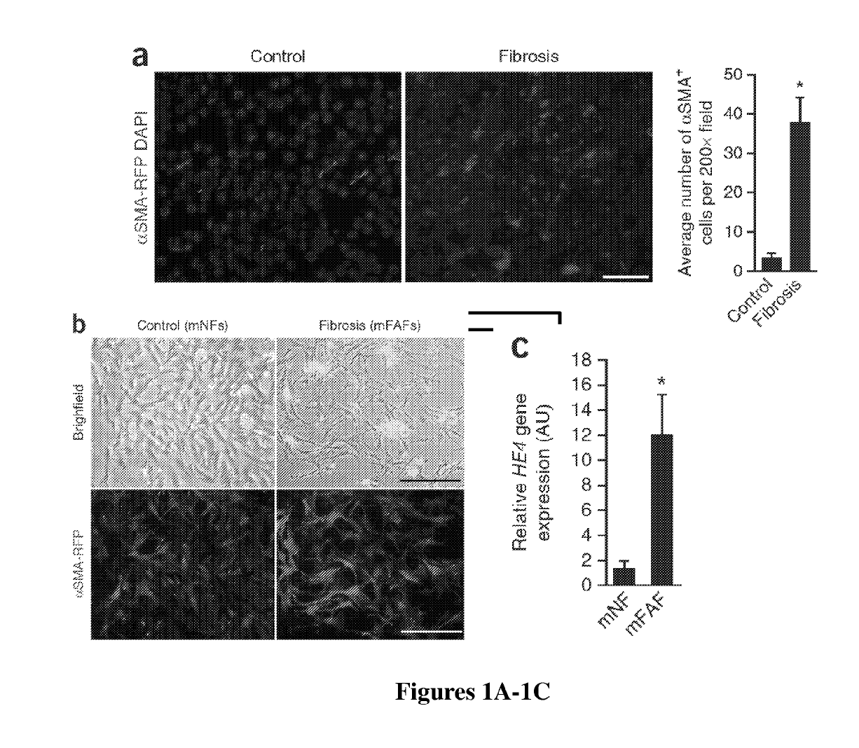

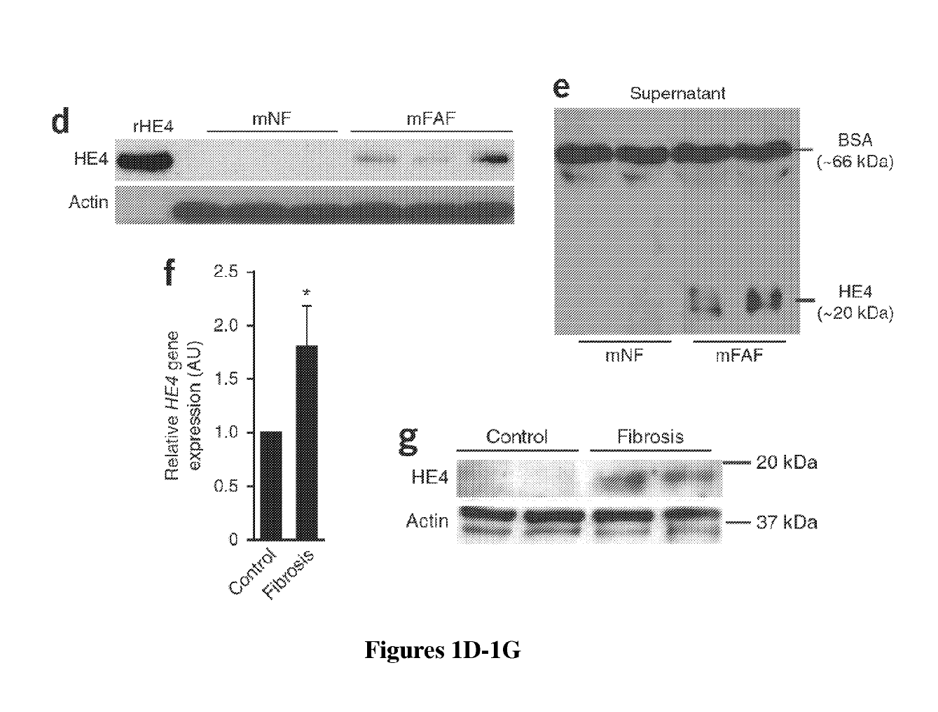

Mice were generated in which the gene for red fluorescent protein (RFP) was expressed under the control of the αSMA (a smooth muscle actin) promoter (referred to here as αSMA-RFP transgenic mice). In healthy kidneys of αSMA-RFP transgenic mice, αSMA+ cells were restricted to occasional rare interstitial cells and smooth muscle cells, whereas a significant increase in the number of interstitial αSMA+ cells (tenfold increase) after unilateral ureteral obstruction (UUO) was found (FIG. 1A). αSMA+ cells were isolated from control and fibrotic kidneys of αSMA-RFP transgenic mice using fluorescence-activated cell sorting, expanded (FIG. 1B) and gene expression profiling was performed to identify candidate genes that may mediate fibrosis. Pathway analysis revealed alterations in genes associated with transforming growth factor β (TGF-β)-mediated cytoskeleton remodeling, mesenchymal phenotype acquisition, cell adhesion and transport of clathrin-coated vesicles. Upregulated genes included those encoding for extracellular matrix proteins, including Bgn (encoding biglycan), Des (encoding desmin) and Dcn (encoding decorin), as well as serine proteases, such as Prss35 and Prss23, and protease inhibitors, including SerpinF1 and Serpina10. Notably, the most highly upregulated gene in this array analysis (with 37-fold increased regulation) was Wfdc2, which encodes for the mouse homolog of HE4 (hereafter “HE4” to indicate both the mouse Wfdc2 and human WFDC2 gene products). Although many of the other top upregulated genes have been previously reported to have a role in liver, lung, colon or kidney fibrosis, the expression profiling data identified HE4 as a new gene with potential implications for fibrosis. Real-time PCR analysis revealed a 12-fold upregulation of HE4 in fibrosis-associated fibroblasts (FAFs) (FIG. 1C). Western blot analyses detected HE4 in FAF lysates and culture media as a single band (FIGS. 1D, 1E). HE4 expression was also found to be elevated in fibrotic kidneys (FIGS. 1F, 1G).

### Example 2

### HE4 is a Pan-Serine Protease and an MMP2 and MMP9 Inhibitor that Prevents Type I Collagen Degradation

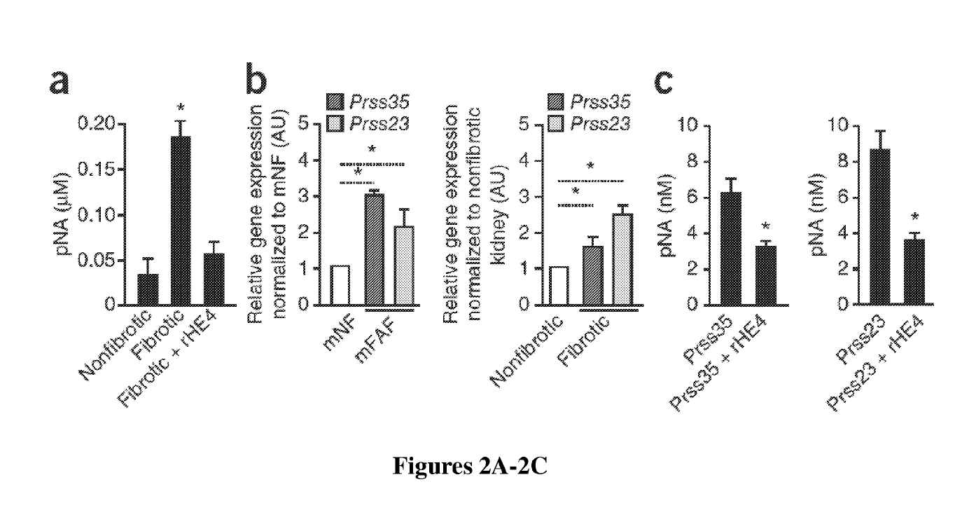

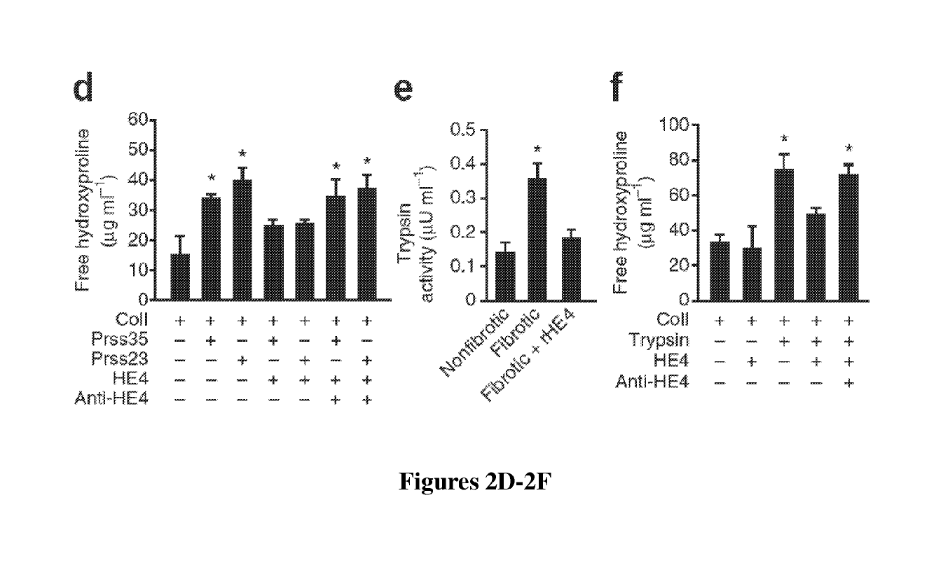

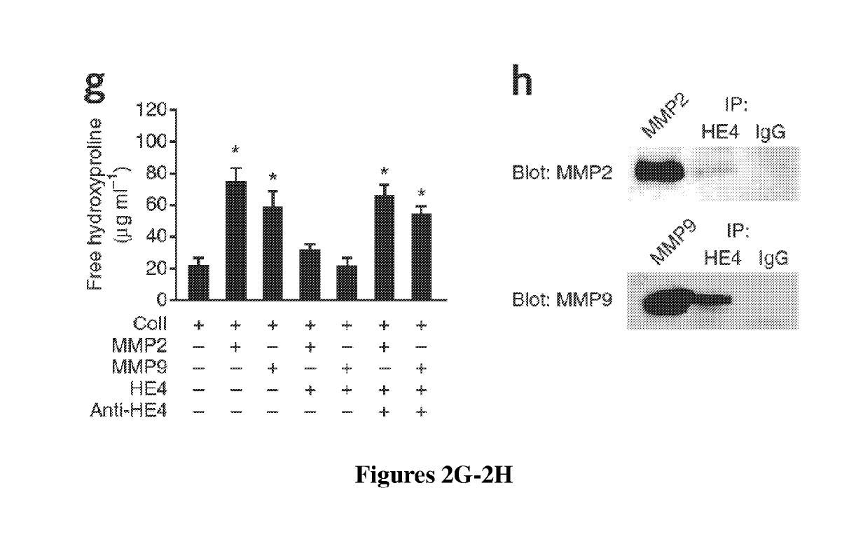

The four-disulfide core domain repeats, or WAP functional motif, of HE4 suggested protease inhibitor activity. Serine protease activity in fibrotic kidney lysates was significantly inhibited when they were preincubated with recombinant HE4 protein (FIG. 2A). In this assay, an increase in the degradation of the substrate BAPNA, measured using spectrophotometric detection of the released p-nitroaniline (pNA) product, indicates an increase in serine protease activity. Addition of HE4 to fibrotic kidney lysates reduced pNA concentrations, which is indicative of its capacity to function as an inhibitor of enzymes with trypsin-like serine protease activity. The FAF and fibrotic kidney gene expression profiles identified the upregulation of two serine proteases with unknown roles in renal fibrosis, Prss35 and Prss23. Validation by real-time PCR revealed 3- and 1.5-fold upregulation of Prss35 and 2.1- and 2.5-fold upregulation of Prss23 in FAFs and fibrotic kidneys, respectively (FIG. 2B). Both Prss23 and Prss35 showed gelatinolytic activity in a zymogram assay, and HE4 specifically inhibited Prss35 and Prss23 serine protease activity and their capacity to degrade type I collagen (FIGS. 2C, 2D). Hydroxyproline release assays measure collagen triple-helix degradation. An increase in released hydroxyproline was found when type I collagen was subjected to either Prss35 or Prss23 serine proteases (FIG. 2D). HE4 inhibited Prss35- and Prss23-mediated degradation of type I collagen (FIG. 2D). Further, the inhibitory activity of HE4 on Prss35 and Prss23 was suppressed by the addition of HE4-neutralizing antibodies (FIG. 2D). HE4 inhibition of trypsin was directly evaluated and HE4 inhibited the enzyme activity of purified trypsin and its activity in fibrotic kidney lysates (FIG. 2E). HE4 also reduced trypsin degradation of type I collagen, and HE4-neutralizing antibodies reversed this action (FIG. 2F). The ability of recombinant human matrix metalloproteinase 2 (MMP2) and MMP9 to degrade type I collagen was evaluated to see if MMP2 and MMP9 could be inhibited by HE4. A hydroxyproline release assay showed that HE4 significantly suppressed the activities of MMP2 and MMP9 to degrade type I collagen (FIG. 2G). Further, it was found that HE4 directly interacts with MMP2 and MMP9, as assessed by immunoprecipitation using antibody to HE4 and subsequent western blotting analysis (FIG. 2H). HE4 also inhibited the degradation of type I collagen by bacterial collagenase, a collagenase that shares an activity profile with many mammalian MMPs.

### Example 3

### HE4 Neutralization Inhibits Kidney Fibrosis

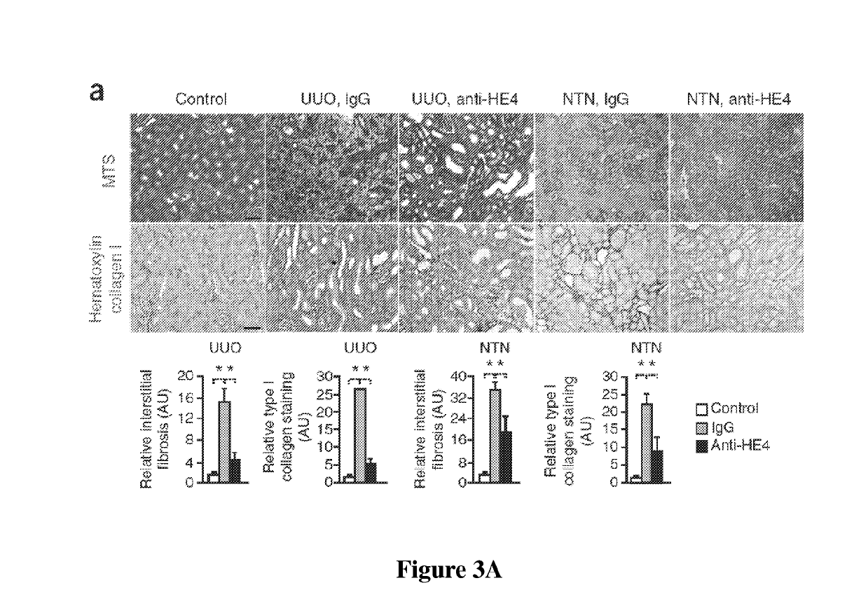

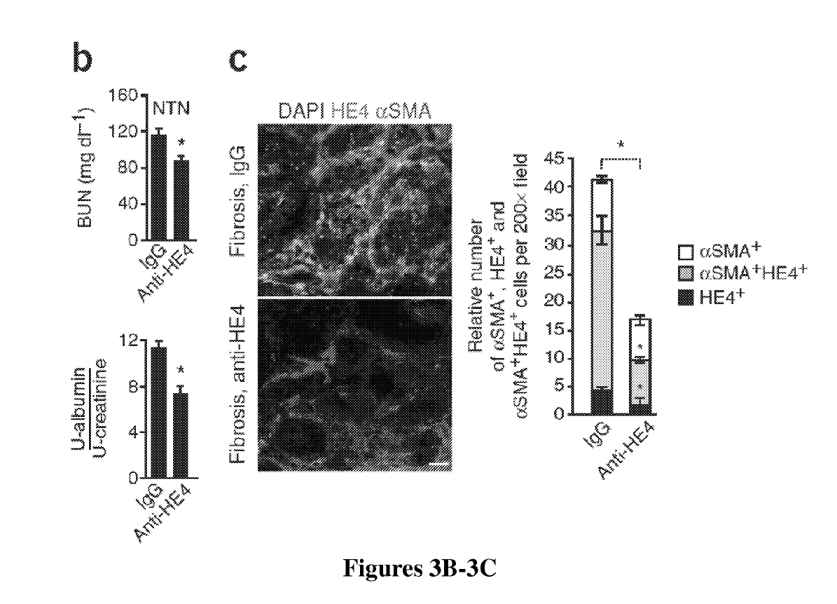

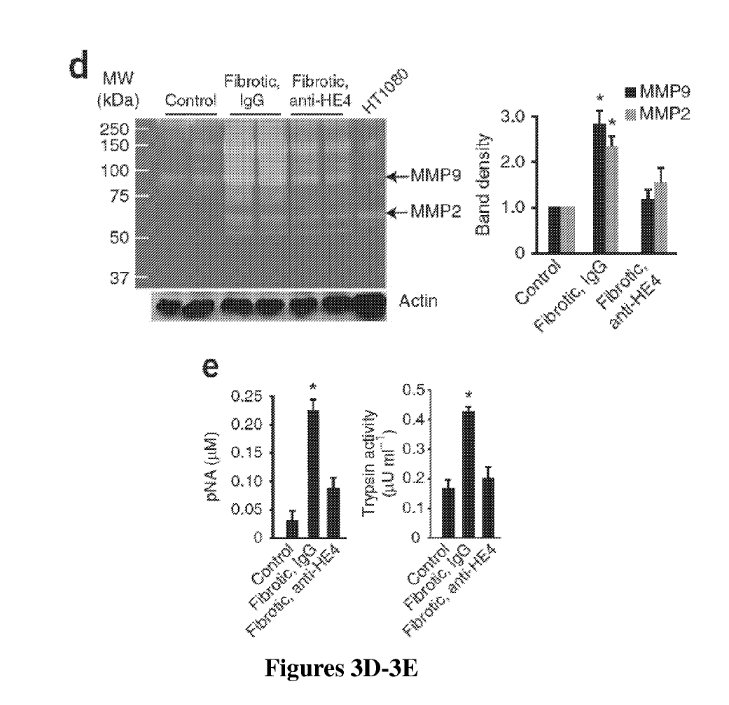

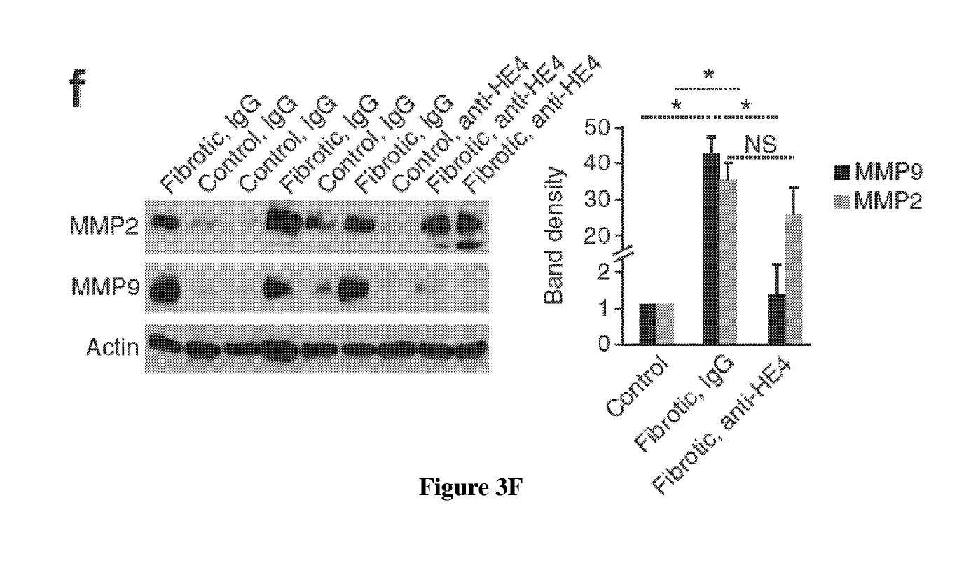

To functionally address the role of HE4 specifically in renal fibrosis, an HE4-neutralizing antibody was administered to mice after UUO. Mice treated with the antibody to HE4 showed improvement in renal fibrosis when compared to mice treated with a control IgG antibody, as shown by a significant decrease in Masson's trichrome staining (75% reduction) and type I collagen content (80% reduction) in the mice treated with the antibody to HE4 (FIG. 3A). In the nephro-toxic serum-induced nephritis (NTN) mouse model, treating mice with the HE4-neutralizing antibody also resulted in a significant reduction in Masson's trichrome staining (50% reduction) and type I collagen content (60% reduction), which is suggestive of reduced renal fibrosis (FIG. 3A). Blood urea nitrogen and urine album in-to-creatinine ratio measurements showed that the treatment with antibody to HE4 improved renal functions in NTN mice (FIG. 3B). Similar results were found in mice with 5/6 nephrectomy. Double immunolabeling for HE4 and αSMA indicated that the majority of HE4+ cells are also αSMA+(˜80%). Mice treated with the antibody to HE4 showed a significant decrease in the number of HE4+αSMA+ double-positive cells compared to mice treated with the control IgG antibody (FIG. 3C). Kidney lysates from UUO mice treated with IgG showed an overall increase in type I collagen digestion activity when compared to kidney lysates from contralateral control kidneys (FIG. 3D). Kidney lysates from UUO mice treated with the antibody to HE4 showed an overall decrease in type I collagen degradation activity, including a decrease in type I collagen digestion mediated by MMP2 and MMP9, as assessed by gelatin zymography (FIG. 3D). Serine protease activity in fibrotic kidney lysates was significantly increased in comparison to nonfibrotic kidney lysates (FIG. 3E). Treatment with HE4-neutralizing antibodies also reduced trypsin and trypsin-like serine protease activity in kidney lysates from treated mice when compared to kidney lysates from IgG-treated control mice (FIG. 3E). These results reflect an overall decrease in protease activity as a measure of improved kidney histology resulting from a reduction in fibrosis in the mice treated with antibody to HE4 at the experimental endpoint (day 10 after UUO). MMP expression was evaluated by western blot analyses of normal and fibrotic mouse kidney lysates. MMP2 and MMP9 were significantly upregulated in the fibrotic kidney lysates (FIG. 3F), and no MMP1, MMP3, MMP8 or MMP12 was detected in the fibrotic kidney lysates by western blotting or gelatin and casein zymography. The amount of MMP9 protein was significantly reduced in kidney lysates from mice treated with the antibody to HE4 (FIG. 3F), again reflecting an overall decrease in fibrosis and improvement in kidney histology in the treated mice rather than a direct effect of treatment with antibody to HE4 on MMPs at the experimental endpoint (day 10 after UUO). In this regard, macrophage infiltration was also reduced in mice treated with the antibody to HE4 as compared to the control mice treated with IgG. Migration, proliferation and αSMA expression of FAFs was not affected by the HE4-neutralizing antibody. Although the number of αSMA+ cells decreased in kidneys from mice treated with the HE4-neutralizing antibody, the relative proportion of proliferating myofibroblasts, as measured by colocalization of αSMA and Ki67, remained similar. These results suggest that the HE4-neutralizing antibody treatment did not directly affect myofibroblast proliferation.

### Example 4

### HE4 is Elevated in Human Fibrotic Kidneys, Human FAFs and Serum of Patients with Renal Fibrosis

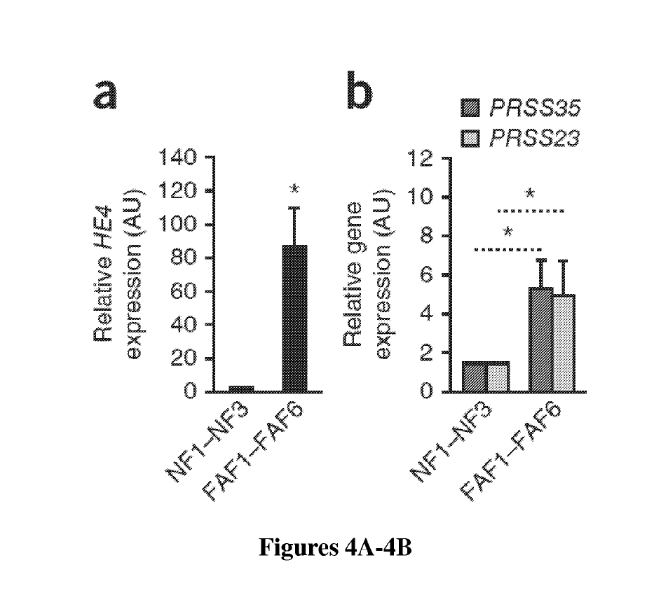

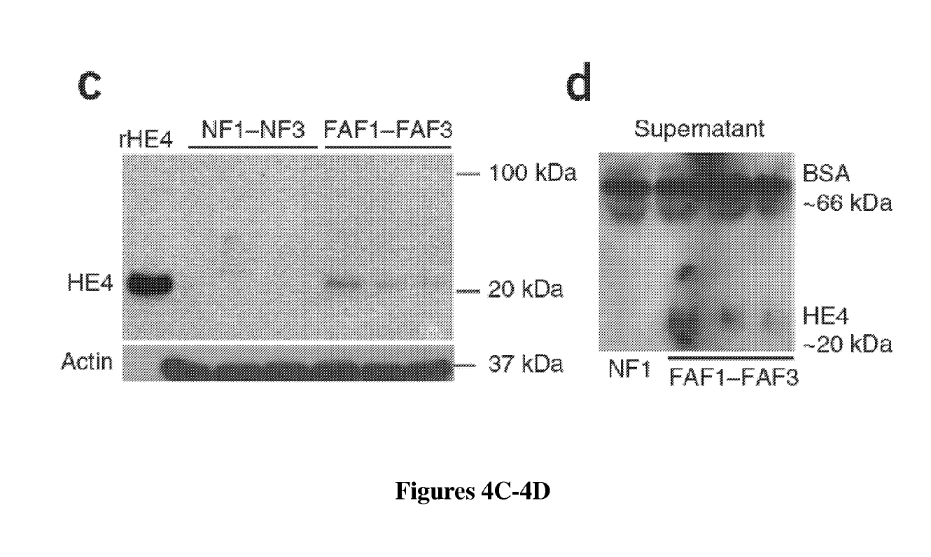

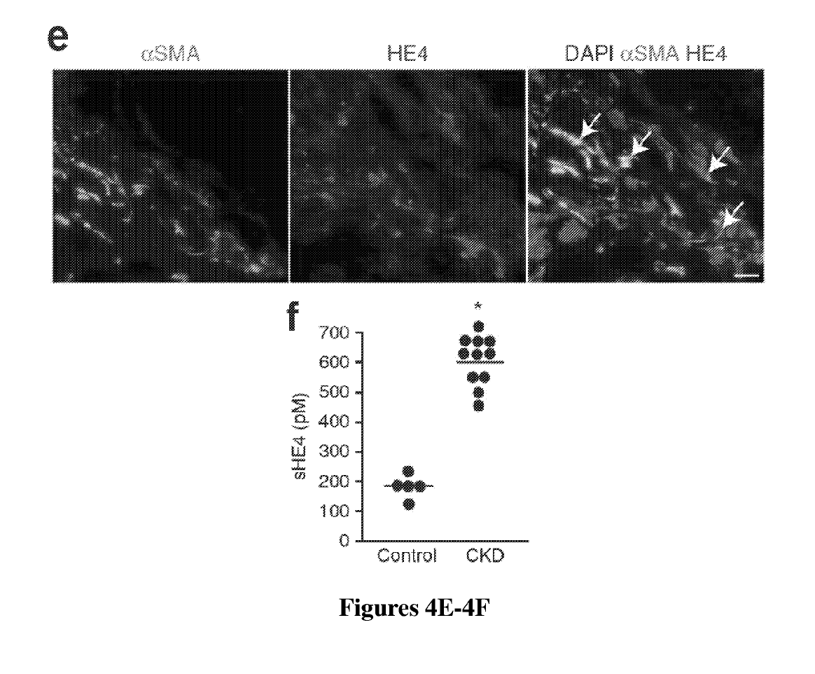

HE4, PRSS35 and PRSS23 was also found upregulated in FAFs from human fibrotic kidneys (FIGS. 4A, 4B). Western blot analyses revealed an upregulation of HE4 in human FAFs (FIG. 4C) and showed that HE4 is secreted by human FAFs (FIG. 4D). Immunolabeling studies further revealed renal interstitial and tubular expression of HE4 in human fibrotic kidneys (FIG. 4E). Additionally, serum concentrations of HE4 were significantly elevated in patients with chronic renal disease with biopsy-confirmed fibrosis (˜600 pM) when compared to control serum from healthy individuals (˜180 pM) (FIG. 4F).

### Other Embodiments

All publications, patent applications, and patents mentioned in this specification are herein incorporated by reference.

Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, pharmacology, or related fields are intended to be within the scope of the invention.

What is claimed is:

1. A method of treating a subject having organ fibrosis, said method comprising administering to said subject an inhibitor of human epididymis protein-4 (HE4) in an amount sufficient to treat said organ fibrosis.

2. A method of treating a subject having organ fibrosis, said method comprising:
a) determining the type I collagen content in a sample from said subject, and
b) administering to a subject having increased type I collagen content an inhibitor of HE4 in an amount sufficient to treat said organ fibrosis.

3. A method of treating a subject having a proliferative disease, said method comprising administering to said subject an inhibitor of HE4 in an amount sufficient to treat said proliferative disease.

4. The method of claim 3, wherein said proliferative disease is selected from the group consisting of: leukemia, brain cancer, bladder cancer, breast cancer, cervical cancer, colorectal cancer, endometrial cancer, esophageal cancer, head and neck cancer, liver cancer, lung cancer, lymphoma, ovarian cancer, pancreatic cancer, prostate cancer, renal cancer, skin cancer, stomach cancer, testis cancer, thyroid cancer, and urothelial cancer.

5. The method of claims 1 and 2, wherein said organ fibrosis is selected from the group consisting of: renal fibrosis, pulmonary fibrosis, cirrhosis, endomyocardial fibrosis, Chrohn's disease, colon fibrosis, liver fibrosis, heart fibrosis, scleroderma, and progressive massive fibrosis.

6. The method of claims 1 and 2, wherein said inhibitor of HE4 is administered in an amount sufficient to further facilitate organ regeneration and repair.

7. The method of claims 1-2, wherein said inhibitor of HE4 results in an increase in serine protease activity in a fibrotic organ.

8. The method of claims 1-3, wherein said inhibitor of HE4 is an RNAi agent, a small molecule inhibitor, or an antibody.

9. The method of claims 1-3, wherein said inhibitor of HE4 is administered with a second agent.

10. The method of claim 9, wherein said second agent is an anticancer agent or an immunosuppressive agent.

11. A method of diagnosing a subject as having, or having a risk of developing organ fibrosis, said method comprising:
a) obtaining a sample from said subject,
b) measuring the level of HE4 in a sample from said subject, and
c) comparing said level to a normal reference sample, wherein an increase in said HE4 levels compared to said normal reference sample results in diagnosing said subject as having, or having a risk of developing organ fibrosis.

12. The method of claim 11, wherein said organ fibrosis is selected from the group consisting of: renal fibrosis, pulmonary fibrosis, cirrhosis, endomyocardial fibrosis, Chrohn's disease, colon fibrosis, and progressive massive fibrosis.

13. The method of claim 11, further comprising measuring the level of Prss23 or Prss35 in a sample from said subject, wherein an increase in said Prss23 or Prss35 levels compared to a normal reference sample results in diagnosing said subject as having, or having a risk of developing organ fibrosis.

14. The method of claim 11, further comprising administering to said subject an inhibitor of HE4, in an amount sufficient to treat said organ fibrosis.

15. The method of claim 14, wherein said inhibitor of HE4 is an RNAi agent, a small molecule inhibitor, or an antibody.

16. The method of claim 14, wherein said inhibitor of HE4 results in an increase in serine protease activity in a fibrotic organ.

17. The method of claim 14, wherein said inhibitor of HE4 is administered with a second agent.

18. The method of claim 17, wherein said second agent is an anticancer agent or an immunosuppressive agent.

19. A method of treating organ fibrosis by administering an inhibitor of HE4 in a subject, said method comprising:
a) determining the level of HE4 in a sample from said subject
b) adjusting the dose of said inhibitor of HE4 in an amount sufficient to treat said organ fibrosis,
wherein an improvement in renal fibrosis measures results in the treatment of said organ fibrosis.

20. The method of claim 19, wherein said improvement in renal fibrosis measures is selected from the group consisting of: a decrease in Masson's Trichrome staining, a decrease in type I collagen content, an increase in type I collagen digestion activity, an increase in serine protease activity, and reduced macrophage infiltration.

21. A method for identifying an inhibitor of HE4, said method comprising contacting a cell with a candidate compound and measuring HE4 activity, wherein the presence of a decrease level of HE4 activity in said cell, as compared to a normal reference sample, identifies an inhibitor of HE4.
